(19) United States
(12) Patent Application Publication
(10) Pub. No.: US 2026/0013832 A1
(43) Pub. Date: Jan. 15, 2026

(54) METHOD FOR GENERATING ULTRASOUND IMAGE AND ULTRASOUND IMAGING APPARATUS

(71) Applicant: Industry-Academic Cooperation Foundation, Dankook University, Gyeonggi-do (KR)

(72) Inventors: HEE CHUL YOON, Seoul (KR); DO YOUNG JANG, Gyeonggi-do (KR)

(21) Appl. No.: 19/077,174

(22) Filed: Mar. 12, 2025

(30) Foreign Application Priority Data
Jul. 10, 2024 (KR) 10-2024-0091114
Nov. 6, 2024 (KR) 10-2024-0156622

(51) Int. Cl. A61B 8/00

(52) U.S. Cl. CPC A61B 8/5207; A61B 8/4483

(57) ABSTRACT

A method for generating an ultrasound image includes obtaining a plurality of sets of received ultrasound data by repeating a process of transmitting ultrasound to a target object with one ultrasound transducer element and receiving ultrasound reflected from the target object with all ultrasound transducer elements using an ultrasound probe including a plurality of ultrasound transducer elements, and generating a plane wave synthetic focused image or a diverging wave synthetic focused image based on the plurality of sets of received ultrasound data.

### CROSS REFERENCE TO RELATED APPLICATION AND CLAIM OF PRIORITY

This application claims the benefit under 35 USC § 119 of Korean Patent Application Nos. 10-2024-0091114 filed on Jul. 10, 2024, and 10-2024-0156622 filed on Nov. 6, 2024, in the Korean Intellectual Property Office, the entire disclosure of which are incorporated herein by reference for all purposes.

### BACKGROUND

### 1. Field

The present disclosure relates to an ultrasound image generation technology.

### 2. Description of Related Art

Ultrasound diagnostic images are currently widely used in clinical practice due to non- invasiveness, real-time diagnosis, high spatial resolution, and stability. In current ultrasound imaging, not only anatomical information of the human body illustrated by B-mode image, which is a conventional ultrasound image, but also functional information of the human body through functional ultrasound imaging technologies such as elasticity imaging and ultrafast Doppler are being utilized for diagnosis.

In particular, ultrafast ultrasound imaging techniques such as plane wave synthetic transmit focusing technique and diverging wave synthetic transmit focusing technique are widely applied to various ultrasound-based functional imaging technologies such as elasticity imaging, ultrafast Doppler, and super-resolution imaging.

### SUMMARY

The disclosed embodiments are intended to provide a method for generating ultrasound image and ultrasound imaging apparatus that generate high-quality ultrasound images without reducing a frame rate.

According to an aspect of the present disclosure, there is provided a method for generating an ultrasound image including obtaining a plurality of sets of received ultrasound data by repeating a process of transmitting ultrasound to a target object with one ultrasound transducer element and receiving ultrasound reflected from the target object with all ultrasound transducer elements using an ultrasound probe including a plurality of ultrasound transducer elements, and generating a plane wave synthetic focused image or a diverging wave synthetic focused image based on the plurality of sets of received ultrasound data.

The generating may include generating a plurality of plane wave reception signals that may be obtained when plane waves are transmitted and received to and from the target object at various angles by analyzing the plurality of sets of received ultrasound data, generating a plane wave synthetic signal by synthesizing the plurality of plane wave reception signals, and generating a plane wave synthetic focused image based on the plane wave synthetic signal.

The generating of the plurality of plane wave reception signals may include calculating a transmission delay time and a reception delay time using Equations 1 and 2 below, and generating a plane wave reception signal having an arbitrary angle that may be obtained when transmitting and receiving a plane wave to the target object at an arbitrary angle based on the transmission delay time and the reception delay time.

$${τ}_{tx} ( x , z , {x}_{n} , {θ}_{s} ) = \frac{( x - {x}_{n} ) sin {θ}_{s} + z cos {θ}_{s}}{c} \qquad [ Equation 1 ]$$

(where, τₜₓ is a transmission delay time, x and z are the x-coordinate and z-coordinate of an image point, respectively, n is an index indicating an ultrasound transmission transducer element, xₙ is the x-coordinate of an ultrasound transmission transducer element n, s is an index indicating a plane wave angle, θₛ is a plane wave angle, and c is speed of sound)

$${τ}_{rx} ( x , z , {x}_{m} ) = \frac{\sqrt{{( x - {x}_{m} )}^{2} + {z}^{2}}}{c} \qquad [ Equation 2 ]$$

(where, τᵣₓ is the reception delay time, m is an index indicating an ultrasound reception transducer element, and xₘ is the x-coordinate of an ultrasound reception transducer element m)

In the generating of the plane wave reception signal having the arbitrary angle, the plane wave reception signal having the arbitrary angle may be generated using Equations 3 and 4 below.

$$τ ( x , z , {x}_{n} , {x}_{m} , {θ}_{s} ) = {τ}_{tx} ( x , z , {x}_{n} , {θ}_{s} ) + {τ}_{rx} ( x , z , {x}_{m} ) \qquad [ Equation 3 ]$$

$${p}_{s} ( x , z ) = {\sum }_{n = 1}^{{N}_{iSF}} {\sum }_{m = 1}^{{N}_{t}} {r}_{n , m} ( τ ( x , z , {x}_{n} , {x}_{m} , {θ}_{s} ) ) \qquad [ Equation 4 ]$$

(where, pₛ is a plane wave reception sound pressure signal, Ni_{SF} is the number of ultrasound transmissions, Nₜ is the total number of ultrasound transducer elements used, and r_{n,m} is a received ultrasound signal obtained by the ultrasound reception transducer element m according to ultrasound transmission of the ultrasound transmission transducer element n)

In the generating of the plane wave synthetic signal, the plane wave synthetic signal may be generated using Equation 5 below.

$$p ( x , z ) = {\sum }_{s = 1}^{{N}_{θ}} {p}_{s} ( x , z ) = {\sum }_{s = 1}^{{N}_{θ}} {\sum }_{n = 1}^{{N}_{iSF}} {\sum }_{m = 1}^{{N}_{t}} {r}_{n , m} ( τ ( x , z , {x}_{n} , {x}_{m} , {θ}_{s} ) ) \qquad [ Equation 5 ]$$

(where, p is the plane wave synthetic signal, and N_{θ} is the total number of arbitrary plane wave angles to be synthesized)

The generating may include generating a plurality of diverging wave reception signals that may be obtained when transmitting and receiving diverging waves generated from virtual sound sources at various locations to and from the target object by analyzing the plurality of sets of received ultrasound data, generating a diverging wave synthetic signal by synthesizing the plurality of diverging wave reception signals, and generating a diverging wave synthetic focused image based on the diverging wave synthetic signal.

The generating of the plurality of diverging wave reception signals may include calculating a transmission delay time and a reception delay time using Equations 6 and 7 below, and generating a diverging wave reception signal at an arbitrary location that may be obtained when transmitting and receiving a diverging wave generated from a virtual sound source at an arbitrary location to and from the target object based on the transmission delay time and the reception delay time.

$${τ}_{tx} ( x , z , {x}_{n} , {x}_{v , s} , {z}_{v , s} ) = \frac{\sqrt{{( x - {x}_{v , s} )}^{2} + {( z - {z}_{v , s} )}^{2}} - \sqrt{{( {x}_{n} - {x}_{v , s} )}^{2} + {( {z}_{v , s} )}^{2}}}{c} \qquad [ Equation 6 ]$$

(where, τₜₓ is a transmission delay time, x and z are the x-coordinate and z-coordinate of an image point, respectively, n is an index indicating an ultrasound transmission transducer element, xₙ is the x-coordinate of an ultrasound transmission transducer element n, s is an index indicating a virtual sound source location, x_{v,s} and z_{v,s} are the x-coordinate and the z-coordinate of the virtual sound source location s, respectively, and c is speed of sound)

$${τ}_{rx} ( x , z , {x}_{m} ) = \frac{\sqrt{{( x - {x}_{m} )}^{2} + {z}^{2}}}{c} \qquad [ Equation 7 ]$$

(wherein, τᵣₓ is a reception delay time, m is an index indicating an ultrasound reception transducer element, and xₘ is the x-coordinate of the ultrasound reception transducer element m)

In the generating of the diverging wave reception signal at an arbitrary location, the diverging wave reception signal at the arbitrary location may be generated using Equations 8 and 9 below.

$${τ}_{tx} ( x , z , {x}_{n} , {x}_{m} , {x}_{v , s} , {z}_{v , s} ) = {τ}_{tx} ( x , z , {x}_{n} , {x}_{v , s} , {z}_{v , s} ) + {τ}_{rx} ( x , z , {x}_{m} ) \qquad [ Equation 8 ]$$

$${p}_{s} ( x , z ) = {\sum }_{n = 1}^{{N}_{iSF}} {\sum }_{m = 1}^{{N}_{t}} {r}_{n , m} ( x , z , {x}_{n} , {x}_{m} , {x}_{v , s} , {z}_{v , s} ) \qquad [ Equation 9 ]$$

(where, pₛ is the diverging wave reception signal, N_{iSF} is the number of ultrasound transmissions, Nₜ is the total number of ultrasound transducer elements used, and r_{n,m} is a received ultrasound signal obtained by the ultrasound reception transducer element m according to ultrasound transmission of the ultrasound transmission transducer element n)

In the generating of the diverging wave synthetic signal, the diverging wave synthetic signal may be generated using Equation 10 below.

$$p ( x , z ) = {\sum }_{s = 1}^{{N}_{v}} {p}_{s} ( x , z ) = {\sum }_{s = 1}^{{N}_{v}} {\sum }_{n = 1}^{{N}_{iSF}} {\sum }_{m = 1}^{{N}_{t}} {r}_{n , m} ( x , z , {x}_{n} , {x}_{m} , {x}_{v , s} , {z}_{v , s} ) \qquad [ Equation 10 ]$$

(where, p is the diverging wave synthesis signal, and N_{θ} is the total number of diverging waves to be synthesized)

According to another aspect of the present disclosure, there is provided an ultrasound imaging apparatus including an ultrasound probe that includes a plurality of ultrasound transducer elements and obtains a plurality of sets of received ultrasound data by repeating a process of transmitting ultrasound to a target object with one ultrasound transducer element and receiving ultrasound reflected from the target object with all ultrasound transducer elements, and an ultrasound image generator configured to generate a plane wave synthetic focused image or a diverging wave synthetic focused image based on the plurality of sets of received ultrasound data.

The ultrasound image generator may be configured to generate a plurality of plane wave reception signals that may be obtained when plane waves are transmitted and received to and from the target object at various angles by analyzing the plurality of sets of received ultrasound data, generate a plane wave synthetic signal by synthesizing the plurality of plane wave reception signals, and generate a plane wave synthetic focused image based on the plane wave synthetic signal.

The ultrasound image generator may be configured to calculate a transmission delay time and a reception delay time using Equations 1 and 2 below and generate a plane wave reception signal having an arbitrary angle that may be obtained when transmitting and receiving a plane wave to the target object at an arbitrary angle based on the transmission delay time and the reception delay time.

$${τ}_{tx} ( x , z , {x}_{n} , {θ}_{s} ) = \frac{( x - {x}_{n} ) sin {θ}_{s} + z cos {θ}_{s}}{c} \qquad [ Equation 1 ]$$

(where, τₜₓ is a transmission delay time, x and z are the x-coordinate and z-coordinate of an image point, respectively, n is an index indicating an ultrasound transmission transducer element, xₙ is the x-coordinate of an ultrasound transmission transducer element n, s is an index indicating a plane wave angle, θₛ is a plane wave angle, and c is speed of sound)

$${τ}_{rx} ( x , z , {x}_{m} ) = \frac{\sqrt{{( x - {x}_{m} )}^{2} + {z}^{2}}}{c} \qquad [ Equation 2 ]$$

(where, τᵣₓ is the reception delay time, m is an index indicating an ultrasound reception transducer element, and xₘ is the x-coordinate of an ultrasound reception transducer element m)

The ultrasound image generator may be configured to generate the plane wave reception signal having the arbitrary angle using Equations 3 and 4 below.

$$τ ( x , z , {x}_{n} , {x}_{m} , {θ}_{s} ) = {τ}_{tx} ( x , z , {x}_{n} , {θ}_{s} ) + {τ}_{rx} ( x , z , {x}_{m} ) \qquad [ Equation 3 ]$$

$${p}_{s} ( x , z ) = {\sum }_{n = 1}^{{N}_{iSF}} {\sum }_{m = 1}^{{N}_{t}} {r}_{n , m} ( τ ( x , z , {x}_{n} , {x}_{m} , {θ}_{s} ) ) \qquad [ Equation 4 ]$$

(where, pₛ is a plane wave reception sound pressure signal, N_{iSF} is the number of ultrasound transmissions, Nₜ is the total number of ultrasound transducer elements used, and r_{n,m} is a received ultrasound signal obtained by the ultrasound reception transducer element m according to ultrasound transmission of the ultrasound transmission transducer element n)

The ultrasound image generator may generate the plane wave synthetic signal using Equation 5 below.

$$p ( x , z ) = {\sum }_{s = 1}^{{N}_{θ}} {p}_{s} ( x , z ) = {\sum }_{s = 1}^{{N}_{θ}} {\sum }_{n = 1}^{{N}_{iSF}} {\sum }_{m = 1}^{{N}_{t}} {r}_{n , m} ( τ ( x , z , {x}_{n} , {x}_{m} , {θ}_{s} ) ) \qquad [ Equation 5 ]$$

(where, p is the plane wave synthetic signal, and N_{θ} is the total number of arbitrary plane wave angles to be synthesized)

The ultrasound image generator may be configured to generate a plurality of diverging wave reception signals that may be obtained when transmitting and receiving diverging waves generated from virtual sound sources at various locations to and from the target object by analyzing the plurality of sets of received ultrasound data, generate a diverging wave synthetic signal by synthesizing the plurality of diverging wave reception signals, and generate a diverging wave synthetic focused image based on the diverging wave synthetic signal.

The ultrasound image generator may be configured to calculate a transmission delay time and a reception delay time using Equations 6 and 7 below and generate a diverging wave reception signal at an arbitrary location that may be obtained when transmitting and receiving a diverging wave generated from a virtual sound source at an arbitrary location to and from the target object based on the transmission delay time and the reception delay time.

$${τ}_{tx} ( x , z , {x}_{n} , {x}_{v , s} , {z}_{v , s} ) = \frac{\sqrt{{( x - {x}_{v , s} )}^{2} + {( z - {z}_{v , s} )}^{2}} - \sqrt{{( {x}_{n} - {x}_{v , s} )}^{2} + {( {z}_{v , s} )}^{2}}}{c} \qquad [ Equation 6 ]$$

(where, τₜₓ is a transmission delay time, x and z are the x-coordinate and z-coordinate of an image point, respectively, n is an index indicating an ultrasound transmission transducer element, xₙ is the x-coordinate of an ultrasound transmission transducer element n, s is an index indicating a virtual sound source location, x_{v,s} and z_{v,s} are the x-coordinate and the z-coordinate of the virtual sound source location s, respectively, and c is speed of sound)

$${T}_{rx} ( x , z , {x}_{m} ) = \frac{\sqrt{{( x - {x}_{m} )}^{2} + {z}^{2}}}{c} \qquad [ Equation 7 ]$$

(wherein, τᵣₓ is a reception delay time, m is an index indicating an ultrasound reception transducer element, and xₘ is the x-coordinate of the ultrasound reception transducer element m)

The ultrasound image generator may be configured to generate the diverging wave reception signal at the arbitrary location using Equations 8 and 9 below.

$$τ ( x , z , {x}_{n} , {x}_{m} , {x}_{v , s} , {z}_{v , s} ) = {τ}_{tx} ( x , z , {x}_{n} , {x}_{v , s} , {z}_{v , s} ) + {τ}_{rx} ( x , z , {x}_{m} ) \qquad [ Equation 8 ]$$

$${p}_{s} ( x , z ) = {\sum }_{n = 1}^{{N}_{iSF}} {\sum }_{m = 1}^{{N}_{t}} {r}_{n , m} ( x , z , {x}_{n} , {x}_{m} , {x}_{v , s} , {z}_{v , s} ) \qquad [ Equation 9 ]$$

(where, pₛ is the diverging wave reception signal, N_{iSF} is the number of ultrasound transmissions, Nₜ is the total number of ultrasound transducer elements used, and r_{n,m} is a received ultrasound signal obtained by the ultrasound reception transducer element m according to ultrasound transmission of the ultrasound transmission transducer element n)

The ultrasound image generator may be configured to generate the diverging wave synthetic signal using Equation 10 below.

$$p ( x , z ) = {\sum }_{s = 1}^{{{N}_{v}}_{}} {p}_{s} ( x , z ) = {\sum }_{s = 1}^{{{N}_{v}}_{}} {\sum }_{{n = 1}^{}}^{{N}_{iSF}} {{\sum }_{m = 1}}^{{N}_{t}} {r}_{n , m} ( x , z , {x}_{n} , {x}_{m} , {x}_{v , s} , {z}_{v , s} ) \qquad [ Equation 10 ]$$

(where, p is the diverging wave synthesis signal, and N_{θ} is the total number of diverging waves to be synthesized).

### BRIEF DESCRIPTION OF THE DRAWINGS

FIG. 1 is a diagram illustrating an ultrasound imaging apparatus according to an exemplary embodiment.

FIG. 2 is an exemplary diagram for describing a method for obtaining a set of received ultrasound data.

FIG. 3 is an exemplary diagram for describing transmission delay and reception delay for generating a plane wave synthetic focused image.

FIG. 4 is an exemplary diagram for describing transmission delay and reception delay for generating a diverging wave synthetic focused image.

FIG. 5 is a diagram illustrating the results of a computer simulation experiment of a method for generating an ultrasound image according to an exemplary embodiment.

FIGS. 6A and 6B are diagrams illustrating the results of a human tissue-mimicking phantom experiment of a method for generating an ultrasound image according to an exemplary embodiment.

FIGS. 7A and 7B are diagrams illustrating the results of a human body experiment of a method for generating an ultrasound image according to an exemplary embodiment.

FIGS. 8A and 8B are exemplary diagrams of a 3D ultrasound computer simulation image generated by a method for generating an ultrasound image according to an exemplary embodiment.

FIG. 9 is a diagram illustrating the results of a method for generating an ultrasound image according to an exemplary embodiment.

FIG. 10 is a diagram illustrating a process of generating a plane wave synthetic focused image according to an exemplary embodiment.

FIG. 11 is a diagram illustrating a process of generating a diverging wave synthetic focused image according to an exemplary embodiment.

FIG. 12 is a block diagram for illustratively describing a computing environment including a computing device according to an exemplary embodiment.

### DETAILED DESCRIPTION

Hereinafter, an embodiment of the present disclosure will be described in detail with reference to the accompanying drawings. In adding reference numerals to components of each drawing, it should be noted that the same components are given the same numerals as much as possible even if they are illustrated in different drawings. In addition, in describing the present disclosure, if it is determined that a specific description of a related known function or configuration may unnecessarily obscure the gist of the present disclosure, the detailed description thereof will be omitted.

The terms described below are terms defined in consideration of the functions in the present disclosure, and may vary depending on the intention or custom of the user or operator. Therefore, the definition should be made based on the contents throughout this specification. Terms such as first, second, etc. may be used to describe various components, but the components should not be limited by the terms. The terms are used only for the purpose of distinguishing one component from another. Singular expressions include plural expressions unless the context clearly indicates otherwise, and terms such as ‘including’ or ‘having’ are intended to specify the presence of features, numbers, steps, operations, components, parts, or combinations thereof described in the specification, but should be understood as not excluding in advance the possibility of the presence or addition of one or more other features, numbers, steps, operations, components, parts, or combinations thereof.

In addition, the division of components in this specification is merely a division by the main function each component is responsible for. That is, two or more components may be combined into one component, or one component may be divided into two or more components with more detailed functions. In addition to the main functions that each component is responsible for, each component may additionally perform some or all of the functions that other components are responsible for, and some of the main functions that each component is responsible for may be performed exclusively by other components. Each component may be implemented by hardware or software, or by a combination of hardware and software.

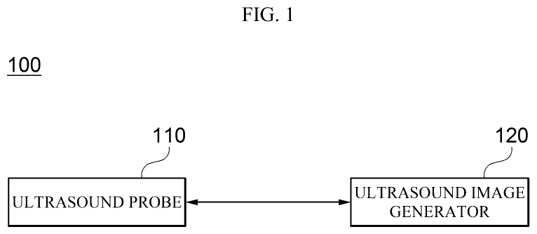

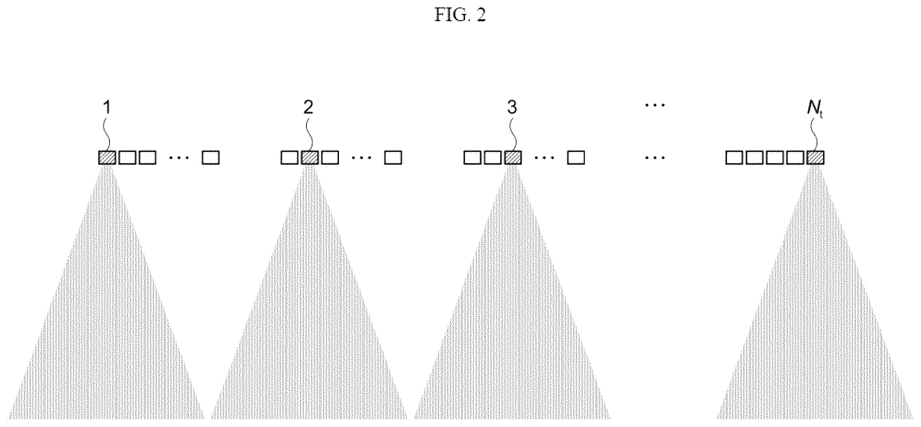

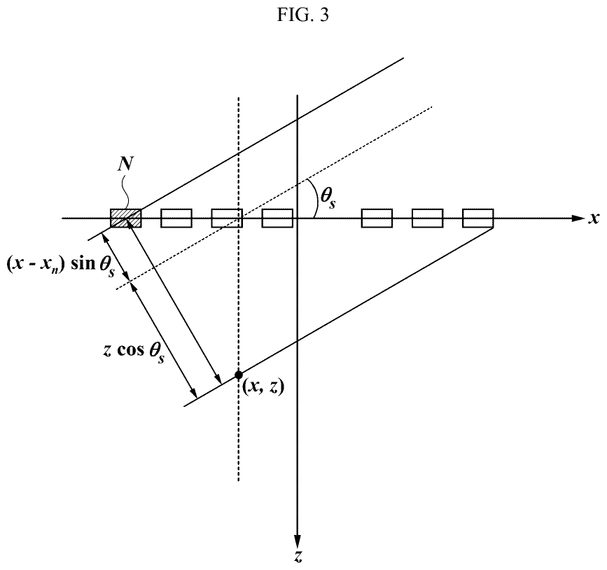

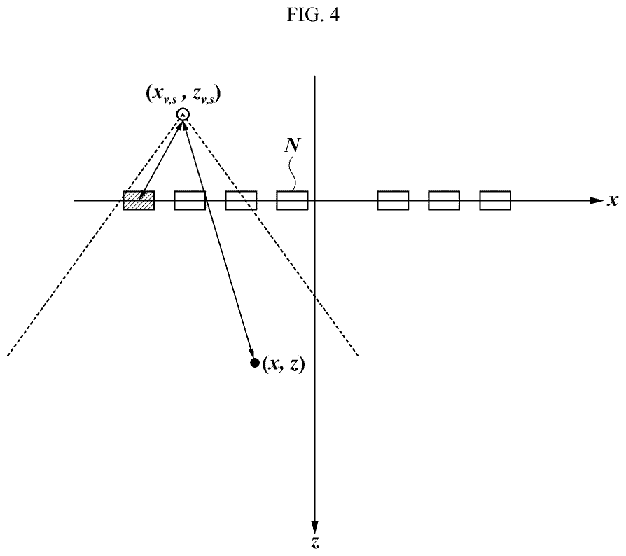

FIG. 1 is a diagram illustrating an ultrasound imaging apparatus according to an exemplary embodiment, FIG. 2 is an exemplary diagram for describing a method for obtaining a set of received ultrasound data, FIG. 3 is an exemplary diagram for describing transmission delay and reception delay for generating a plane wave synthetic focused image, and FIG. 4 is an exemplary diagram for describing transmission delay and reception delay for generating a diverging wave synthetic focused image.

An ultrasound imaging apparatus 100 may be implemented as a portable or cart type. The portable ultrasound imaging apparatus 100 may include, but is not limited to, a smartphone, a laptop computer, a tablet PC, etc. including an ultrasound probe 110 and an application.

Referring to FIG. 1, the ultrasound imaging apparatus 100 according to an exemplary embodiment may include the ultrasound probe 110 and an ultrasound image generator 120.

The ultrasound probe 110 may include a plurality of ultrasound transducer elements. The plurality of ultrasound transducer elements may be arranged one-dimensionally or two-dimensionally.

The ultrasound probe 110 may transmit an ultrasound signal to a target object according to a predetermined control signal and receive an ultrasound signal reflected from the target object.

Specifically, the ultrasound probe 110 may transmit an ultrasound signal to the target object with one ultrasound transducer element according to a predetermined control signal and receive an ultrasound signal reflected from the target object with all ultrasound transducer elements to obtain one set of received ultrasound data. In addition, the ultrasound probe 110 may obtain a plurality of sets of received ultrasound data by repeating the process described above while changing the ultrasound transducer element that transmits the ultrasound signal.

For example, assuming that the ultrasound probe 110 includes Nₜ ultrasound transducer elements 1, 2, 3, . . . , and Nₜ as illustrated in FIG. 2, the ultrasound probe 110 may transmit an ultrasound wave to the target object using the first ultrasound transducer element 1 and receives an ultrasound wave reflected from the target object with all ultrasound transducer elements 1, 2, 3, . . . , and Nₜ to obtain a first set of received ultrasound data, transmit an ultrasound wave to the target object using the second ultrasound transducer element 2 and receives an ultrasound wave reflected from the target object with all ultrasound transducer elements 1, 2, 3, . . . , and Nₜ to obtain a second set of received ultrasound data, and transmit an ultrasound wave to the target object using the second ultrasound transducer element 2 and receives an ultrasound wave reflected from the target object with all ultrasound transducer elements 1, 2, 3, . . . , and Nₜ to obtain a third set of received ultrasound data. In this way, Nₜ sets of received ultrasound data may be obtained by repeating ultrasound transmission and reception while changing the ultrasound transmission transducer element.

The ultrasound probe 110 may be a wired or wireless ultrasound probe.

The ultrasound image generator 120 may generate a plane wave synthetic focused image or a diverging wave synthetic focused image based on the obtained plurality of sets of received ultrasound data.

### Plane Wave Synthetic Focused Image Generation

The ultrasound image generator 120 generates a plurality of plane wave reception signals that may be obtained when plane waves are transmitted and received to and from the target object at various angles by analyzing the plurality of sets of received ultrasound data.

Specifically, the ultrasound image generator 120 may calculate a transmission delay time and a reception delay time of the ultrasound waves for generating a plane wave reception signal that may be obtained when a plane wave is transmitted and received to and from a target object at an arbitrary angle.

For example, referring to FIG. 3, assuming that a plane wave is transmitted and received at an arbitrary angle θₛ, when an ultrasound wave is transmitted from an arbitrary ultrasound transducer element Nₙ and an ultrasound wave is received from an arbitrary ultrasound transducer element Nₘ, a transmission delay time tix and a reception delay time τᵣₓ of the ultrasound wave may be expressed by Equations 1 and 2.

$${τ}_{tx} ( x , z , {x}_{n} , {θ}_{s} ) = \frac{( x - {x}_{n} ) sin {θ}_{s} + z cos {θ}_{s}}{c} \qquad [ Equation 1 ]$$

Here, x and z are the x-coordinate and z-coordinate of an image point, respectively, n is an index indicating an ultrasound transmission transducer element, xₙ is the x-coordinate of an ultrasound transmission transducer element n, s is an index indicating a plane wave angle, θₛ is a plane wave angle, and c is speed of sound.

$${τ}_{rx} ( x , z , {x}_{m} ) = \frac{\sqrt{{( x - {x}_{m} )}^{2} + {z}^{2}}}{c} \qquad [ Equation 2 ]$$

Here, m is an index indicating an ultrasound reception transducer element, and xₘ is the x-coordinate of an ultrasound reception transducer element m.

The ultrasound image generator 120 may generate a plane wave reception signal that may be obtained when transmitting and receiving a plane wave to the target object at an arbitrary angle based on the calculated transmission delay time and reception delay time. For example, the ultrasound image generator 120 may generate a plane wave reception signal pₛ at an arbitrary angle using Equations 3 and 4.

$$τ ( x , z , {x}_{n} , {x}_{m} , {θ}_{s} ) = {τ}_{tx} ( x , z , {x}_{n} , {θ}_{s} ) + {τ}_{rx} ( x , z , {x}_{m} ) \qquad [ Equation 3 ]$$

Here, τ may be a transmission and reception delay time.

$${p}_{s} ( x , z ) = {{\sum }_{n = 1}}^{{N}_{iSF}} {\sum }_{m = 1}^{{N}_{t}} {r}_{n , m} ( τ ( x , z , {x}_{n} , {x}_{m} , {θ}_{s} ) ) \qquad [ Equation 4 ]$$

Here, N_{iSF} is the number of ultrasound transmissions, N, is the total number of ultrasound transducer elements used, n is an index indicating an ultrasound transmission transducer element, and r_{n,m} is a received ultrasound signal obtained by the ultrasound reception transducer element m according to ultrasound transmission of the ultrasound transmission transducer element n.

The ultrasound image generator 120 may generate a plane wave synthetic signal by synthesizing the plurality of plane wave reception signals, and generate a plane wave synthetic focused image based on the generated plane wave synthetic signal.

In this case, the ultrasound image generator 120 may generate a plane wave synthesis signal p using Equation 5.

$$p ( x , z ) = {\sum }_{s = 1}^{{N}_{θ}} {p}_{s} ( x , z ) = {{\sum }_{s = 1}}^{{N}_{θ}} {\sum }_{n = 1}^{{N}_{iSF}} {\sum }_{m = 1}^{{N}_{t}} {r}_{n , m} ( τ ( x , z , {x}_{n} , {x}_{m} , {θ}_{s} ) ) \qquad [ Equation 5 ]$$

Here, s is an index indicating a plane wave angle, and N_{θ} may be the total number of arbitrary plane wave angles to be synthesized.

The plane wave synthetic transmit focusing technique or plane wave synthetic focusing technique is a technique for synthesizing signals transmitted and received at various angles of plane waves. In order to improve image quality in this technique, it is necessary to increase the number of plane wave angles to be synthesized. However, increasing the number of plane wave angles causes a problem of reducing the frame rate. The ultrasound image generator 120 can generate plane wave reception signals at innumerable arbitrary plane wave angles, which can be obtained by the plane wave synthetic transmit focusing technique by repeating the process of transmitting ultrasound with one ultrasound transducer element and receiving ultrasound with all ultrasound transducer elements to obtain and analyze a plurality of sets of received ultrasound data, thereby generating high-quality ultrasound images without reducing the frame rate.

### Diverging Wave Synthetic Focused Image Generation

The ultrasound image generator 120 may generate a plurality of diverging wave reception signals that may be obtained when transmitting and receiving diverging waves generated from virtual sound sources at various locations to and from the target object by analyzing the plurality of sets of received ultrasound data.

Specifically, the ultrasound image generator 120 may calculate the transmission delay time and reception delay time of ultrasound waves for generating diverging wave reception signals that may be obtained when transmitting and receiving diverging waves generated from virtual sound sources at arbitrary locations to and from the target object.

For example, referring to FIG. 4, assuming that diverging waves generated from virtual sound sources at arbitrary locations are transmitted and received, when an ultrasound wave is transmitted from an arbitrary ultrasound transducer element Nₙ and an ultrasound wave is received by an arbitrary ultrasound transducer element Nₘ, the transmission delay time τₜₓ and the reception delay time τᵣₓ of the ultrasound wave may be expressed by Equations 6 and 7.

$${τ}_{tx} ( x , z , {x}_{n} , {x}_{v , s} , {z}_{v , s} ) = \frac{\sqrt{{( x - {x}_{v , s} )}^{2} + {( z - {z}_{v , s} )}^{2}} - \sqrt{{( {x}_{n} - {x}_{v , s} )}^{2} + {( {z}_{v , s} )}^{2}}}{c} \qquad [ Equation 6 ]$$

Here, x and z are the x-coordinate and z-coordinate of an image point, respectively, n is an index indicating an ultrasound transmission transducer element, xₙ is the x-coordinate of an ultrasound transmission transducer element n, s is an index indicating a virtual sound source location, x_{v,s} and z_{v,s} are the x-coordinate and the z-coordinate of the virtual sound source location s, respectively, and c is speed of sound.

$${τ}_{rx} ( x , z , {x}_{m} ) = \frac{\sqrt{{( x - {x}_{m} )}^{2} + {z}^{2}}}{c} \qquad [ Equation 7 ]$$

Here, m is an index indicating an ultrasound reception transducer element, and xₘ is the x-coordinate of the ultrasound reception transducer element m.

The ultrasound image generator 120 may generate a diverging wave reception signal that may be obtained when transmitting and receiving a diverging wave generated from a virtual sound source at an arbitrary location to and from the target object based on the calculated transmission delay time and reception delay time.

For example, the ultrasound image generator 120 may generate a diverging wave reception signal pₛ at an arbitrary angle using Equations 8 and 9.

$$τ ( x , z , {x}_{n} , {x}_{m} , {x}_{v , s} , {z}_{v , s} ) = {τ}_{tx} ( x , z , {x}_{n} , {x}_{v , s} , {z}_{v , s} ) + {τ}_{rx} ( x , z , {x}_{m} ) \qquad [ Equation 8 ]$$

Here, τ may be a transmission/reception delay time.

$${p}_{s} ( x , z ) = {\sum }_{n = 1}^{{N}_{iSF}} {{\sum }^{{N}_{t}}}_{m = 1} {r}_{n , m} ( x , z , {x}_{n} , {x}_{m} , {x}_{v , s} , {z}_{v , s} ) \qquad [ Equation 9 ]$$

Here, N_{iSF} is the number of ultrasound transmissions, N, is the total number of ultrasound transducer elements used, n is an index indicating an ultrasound transmission transducer element, m is an index indicating an ultrasound reception transducer element, and r_{n,m} is a received ultrasound signal obtained by the ultrasound reception transducer element m according to ultrasound transmission of the ultrasound transmission transducer element n.

The ultrasound image generator 120 may generate a diverging wave synthesis signal by synthesizing a plurality of diverging wave synthetic signals at various virtual sound source locations, and generate a diverging wave synthetic focused image based on the generated diverging wave synthesis signal.

In this case, the ultrasound image generator 120 may generate the diverging wave synthesis signal p using Equation 10.

$$p ( x , z ) = {\sum }_{s = 1}^{{{N}_{v}}_{}} {p}_{s} ( x , z ) = {\sum }_{s = 1}^{{{N}_{v}}_{}} {\sum }_{{n = 1}^{}}^{{N}_{iSF}} {{\sum }_{m = 1}}^{{N}_{t}} {r}_{n , m} ( x , z , {x}_{n} , {x}_{m} , {x}_{v , s} , {z}_{v , s} ) \qquad [ Equation 10 ]$$

Here, s is an index indicating the virtual sound source location, and Nᵥ may be any virtual sound source to be synthesized, i.e., the total number of arbitrary diverging waves.

The diverging wave synthetic transmit focusing technique or diverging wave synthetic focusing technique is a technique for synthesizing signals obtained by transmitting and receiving diverging signals generated from a virtual sound source at an arbitrary location. In order to improve image quality in this technique, it is necessary to increase the number of virtual sound sources, i.e., diverging waves, to be synthesized. However, increasing the number of virtual sound sources, i.e., diverging waves, causes a problem of reducing the frame rate. The ultrasound imaging apparatus 100 according to one embodiment can generate diverging wave reception signals for innumerable arbitrary virtual sound source locations, which can be obtained by the diverging wave synthetic transmit focusing technique or the diverging wave synthetic focusing technique, by repeating the process of transmitting ultrasound with one ultrasound transducer element and receiving ultrasound with all ultrasound transducer elements, and obtaining and analyzing to obtain and analyze a plurality of sets of received ultrasound data, thereby generating high-quality ultrasound images without reducing the frame rate.

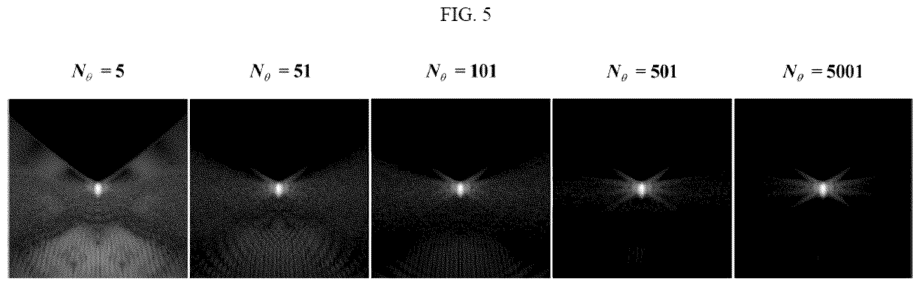

FIG. 5 is a diagram illustrating the results of a computer simulation experiment of a method for generating an ultrasound image according to an exemplary embodiment.

FIG. 5 shows ultrasound images (plane wave synthetic focused images) generated when an image dynamic range is 80 dB, the number of ultrasound transducer elements is 128, a plane wave angle range is −25° to 25°, and the number of plane wave angles N_{θ} is 5, 51, 101, 501, and 5001, respectively.

Referring to FIG. 5, it can be confirmed from the computer simulation results that as the number of plane wave angles N_{θ} increases, a higher quality ultrasound image can be generated. In particular, when the number of plane wave angles N_{θ} at which the highest quality ultrasound image is generated is N_{θ}=5001, 5001 ultrasound transmissions and receptions are required using a conventional plane wave synthetic transmit focusing technique or plane wave synthetic focusing technique. However, it can be confirmed that the method for generating the ultrasound image according to the exemplary embodiment may generate an ultrasound image with only the total number of ultrasound transducer elements, that is, 128 ultrasound transmissions and receptions.

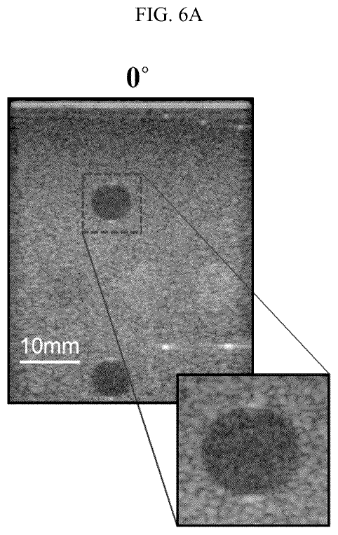

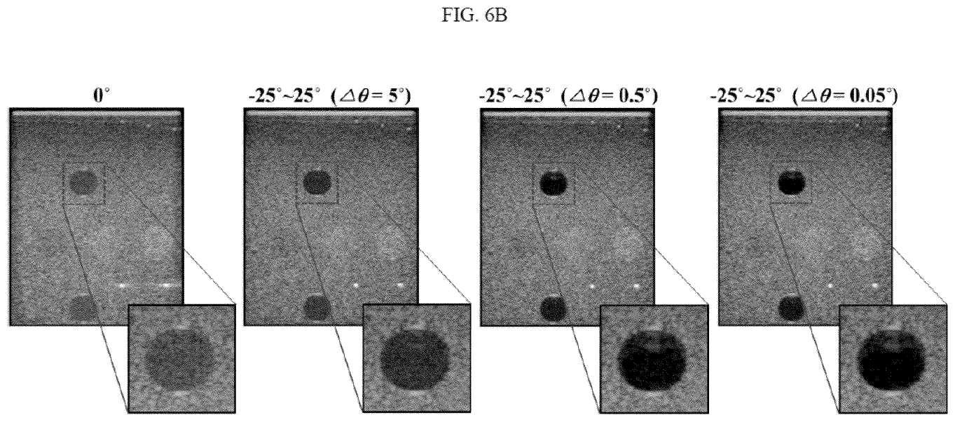

FIGS. 6A and 6B are diagrams illustrating the results of a human tissue-mimicking phantom experiment of the method for generating the ultrasound image according to an exemplary embodiment. FIG. 6A is an ultrasound image generated by the conventional plane wave synthetic transmit focusing or plane wave synthetic focusing technique, and FIG. 6B is an ultrasound image generated by the method for generating the ultrasound image according to an exemplary embodiment. Specifically, FIG. 6A illustrates an ultrasound image generated by the conventional plane wave synthetic transmit focusing technique or plane wave synthetic focusing technique when the plane wave angle is 0°, and FIG. 6B illustrates ultrasound images that are respectively generated when the plane wave angle is 0° (the number of plane wave angles is 1), when the plane wave angle changes by 5° in a range of −25° to 25° (the number of plane wave angles is 11), when the plane wave angle changes by 0.5° in the range of −25° to 25° (the number of plane wave angles is 101), and when the plane wave angle changes by 0.05° in the range of −25° to 25° (the number of plane wave angles is 1001) by the method for generating the ultrasound image according to an exemplary embodiment

Referring to FIGS. 6A and 6B, it can be confirmed that as the results of the human body simulation experiment, under the same conditions, the method for generating the ultrasound image according to an exemplary embodiment can generate an ultrasound image having similar quality to the conventional technique.

In addition, it can be confirmed that the quality of the ultrasound image generated by the method for generating the ultrasound image according to the exemplary embodiment improves as the number of plane wave angles increases.

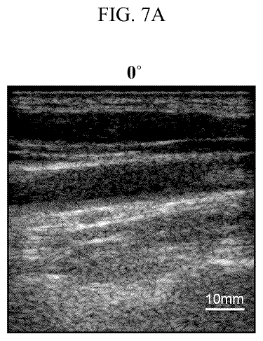

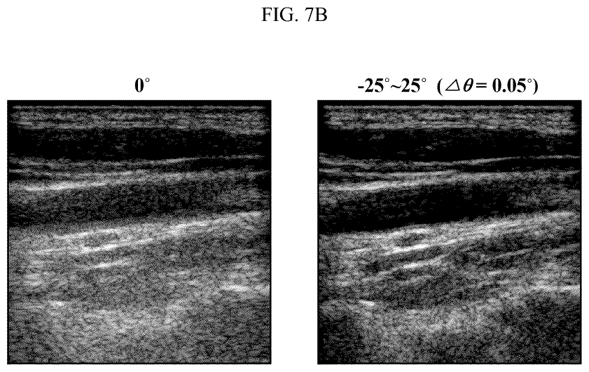

FIGS. 7A and 7B are diagrams illustrating the results of a human body experiment of the method for generating the ultrasound image according to the exemplary embodiment. FIG. 7A illustrates an ultrasound image generated by the conventional plane wave synthetic transmit focusing technique or plane wave synthetic focusing technique, and FIG. 7B illustrates an ultrasound image generated by the method for generating an ultrasound image according to the exemplary embodiment. Specifically, FIG. 7A illustrates an ultrasound image generated by the conventional plane wave synthetic transmit focusing technique or plane wave synthetic focusing technique when the plane wave angle is 0°, and FIG. 7B illustrates ultrasound images generated when the plane wave angle is 0° (the number of plane wave angles is 1) and when the plane wave angle changes by 0.05° in the range of −25° to 25° (the number of plane wave angles is 1001) that are generated by the method for generating the ultrasound image according to the exemplary embodiment.

Referring to FIGS. 7A and 7B, it can be confirmed that, as the human body experiment results, under the same conditions, the method for generating the ultrasound image according to the exemplary embodiment can generate an ultrasound image having similar quality to the conventional technique.

In addition, it can be confirmed that the quality of an ultrasound image generated by the method for generating the ultrasound image according to an exemplary embodiment has better quality when the number of plane wave angles is 1001 compared to when the number of plane wave angles is 1.

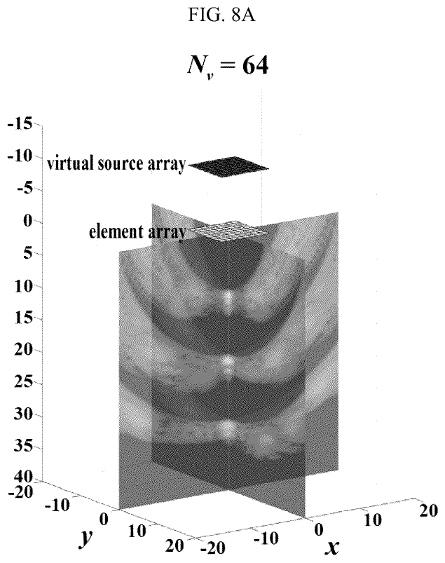

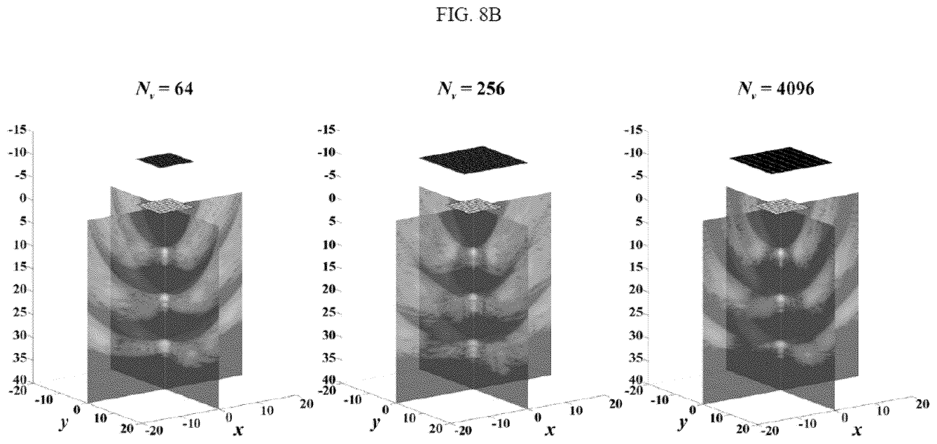

FIGS. 8A and 8B are exemplary diagrams of a 3D ultrasound computer simulation experiment image generated by a method for generating an ultrasound image according to an exemplary embodiment. FIG. 8A is a 3D ultrasound image generated by a conventional diverging wave synthetic transmit focusing or diverging wave synthetic focusing technique, and FIG. 8B is a 3D ultrasound image generated by a method for generating an ultrasound image according to an exemplary embodiment. Specifically, FIG. 8A illustrates a 3D ultrasound image generated by the conventional diverging wave synthetic transmit focusing or diverging wave synthetic focusing technique using an 8*8 virtual sound source, and FIG. 8B illustrates a 3D ultrasound image generated by the method for generating the ultrasound image according to an exemplary embodiment using 8*8 virtual sound sources (Nᵥ=64), 16*16 virtual sound sources (Nᵥ=256), and 64*64 virtual sound sources (Nᵥ=4096), respectively.

Referring to FIGS. 8A and 8B, it can be confirmed that, under the same conditions, a 3D ultrasound image having similar quality to the conventional technique can be generated by the method for generating the ultrasound image according to the exemplary embodiment.

In addition, it can be confirmed that the quality of the 3D ultrasound image generated by the method for generating the ultrasound image according to the exemplary embodiment improves as the number of virtual sound sources increases.

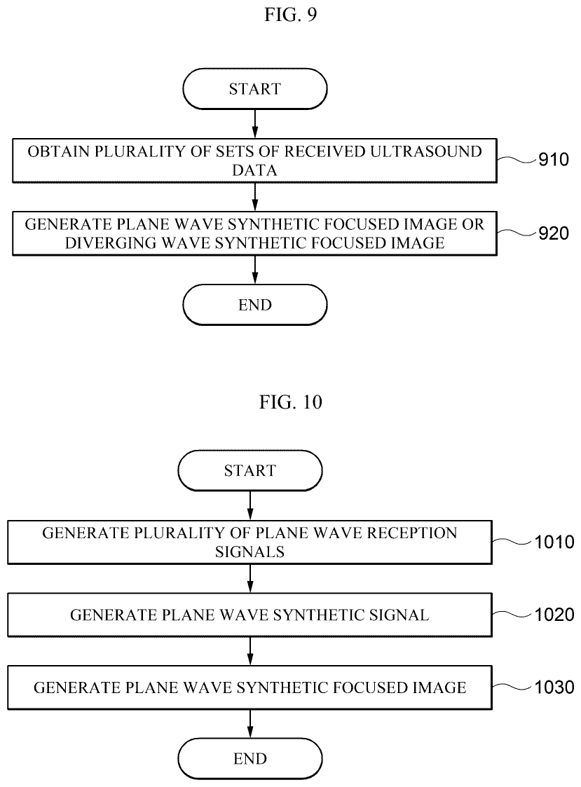

FIG. 9 is a diagram illustrating a method for generating an ultrasound image according to an exemplary embodiment.

The ultrasound image generation method of FIG. 9 may be performed by the ultrasound imaging apparatus 100 of FIG. 1. In the illustrated flowchart, the method for generating the ultrasound image is described by dividing the method into a plurality of steps, but at least some of the steps may be performed in a different order, performed together with other steps by being combined therewith, omitted, performed by being divided into sub-steps, or performed by being added with one or more steps (not illustrated).

Referring to FIG. 9, in step 910, the ultrasound imaging apparatus may obtain a plurality of sets of received ultrasound data by repeating the process of transmitting ultrasound to the target object with one ultrasound transducer element and receiving ultrasound reflected from the target object with all ultrasound transducer elements.

In step 920, the ultrasound imaging apparatus may generate a plane wave synthetic focused image or a diverging wave synthetic focused image based on the obtained plurality of sets of received ultrasound data.

FIG. 10 is a diagram illustrating a process of generating the plane wave synthetic focused image according to an exemplary embodiment. The process of generating the plane wave synthetic focused image of FIG. 10 may be an embodiment of step 920 of FIG. 9.

Referring to FIG. 10, in step 1010, the ultrasound imaging apparatus may generate a plurality of plane wave reception signals that may be obtained when plane waves are transmitted and received to and from a target object at various angles by analyzing a plurality of sets of received ultrasound data.

For example, the ultrasound imaging apparatus may calculate the transmission delay time and the reception delay time of the ultrasound using Equations 1 and 2 described above, and generate a plurality of plane wave reception signals that may be obtained when transmitting and receiving plane waves to and from the target object at various angles using Equations 3 and 4.

In step 1020, the ultrasound imaging apparatus may generate a plane wave synthetic signal by synthesizing a plurality of plane wave reception signals at various angles.

For example, the ultrasound imaging apparatus may generate a plane wave synthetic signal using Equation 5.

In step 1030, the ultrasound imaging apparatus may generate a plane wave synthetic focused image based on the generated plane wave synthetic signal.

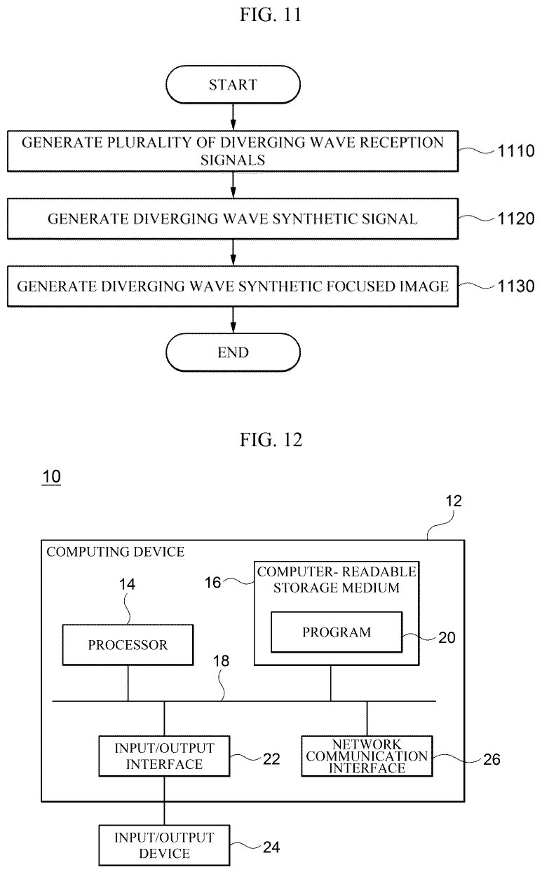

FIG. 11 is a diagram illustrating a process of generating a diverging wave synthetic focused image according to an exemplary embodiment. The diverging wave synthetic focused image generating process of FIG. 11 may be an embodiment of step 920 of FIG. 9.

Referring to FIG. 11, in step 1110, the ultrasound imaging apparatus may generate a plurality of diverging wave reception signals that may be obtained when transmitting and receiving diverging waves generated from virtual sound sources at various locations to and from a target object by analyzing a plurality of sets of received ultrasound data.

For example, the ultrasound imaging apparatus may calculate the transmission delay time and the reception delay time of the ultrasound waves using Equations 6 and 7, and generate the plurality of diverging wave reception signals that may be obtained when transmitting and receiving diverging waves generated from virtual sound sources at various locations to and from a target object using Equations 8 and 9.

In step 1120, the ultrasound imaging apparatus may generate a diverging wave synthetic signal by synthesizing the plurality of diverging wave synthetic signals at various virtual sound source locations.

For example, the ultrasound imaging apparatus may generate a diverging wave synthetic signal using Equation 10.

In step 1130, the ultrasound imaging apparatus may generate a diverging wave synthetic focused image based on the generated diverging wave synthetic signal.

FIG. 12 is a block diagram illustrating a computing environment including a computing device according to an exemplary embodiment. In the illustrated embodiment, respective components may have different functions and capabilities other than those described below, and may include additional components in addition to those described below.

An illustrated computing environment 10 includes a computing device 12. The computing device 12 may be one or more components included in the ultrasound imaging apparatus 100 according to one embodiment.

The computing device 12 includes at least one processor 14, a computer-readable storage medium 16, and a communication bus 18. The processor 14 may cause the computing device 12 to operate according to the exemplary embodiment described above. For example, the processor 14 may execute one or more programs stored on the computer-readable storage medium 16. The one or more programs may include one or more computer-executable instructions, which, when executed by the processor 14, may be configured so that the computing device 12 performs operations according to the exemplary embodiment.

The computer-readable storage medium 16 is configured to store the computer-executable instruction or program code, program data, and/or other suitable forms of information. A program 20 stored in the computer-readable storage medium 16 includes a set of instructions executable by the processor 14. In an embodiment, the computer-readable storage medium 16 may be a memory (volatile memory such as a random access memory, non-volatile memory, or any suitable combination thereof), one or more magnetic disk storage devices, optical disk storage devices, flash memory devices, other types of storage media that are accessible by the computing device 12 and capable of storing desired information, or any suitable combination thereof.

The communication bus 18 interconnects various other components of the computing device 12, including the processor 14 and the computer-readable storage medium 16.

The computing device 12 may also include one or more input/output interfaces 22 that provide an interface for one or more input/output devices 24, and one or more network communication interfaces 26. The input/output interface 22 and the network communication interface 26 are connected to the communication bus 18. The input/output device 24 may be connected to other components of the computing device 12 through the input/output interface 22. The exemplary input/output device 24 may include a pointing device (such as a mouse or trackpad), a keyboard, a touch input device (such as a touch pad or touch screen), a speech or sound input device, input devices such as various types of sensor devices and/or photographing devices, and/or output devices such as a display device, a printer, a speaker, and/or a network card. The exemplary input/output device 24 may be included inside the computing device 12 as a component configuring the computing device 12, or may be connected to the computing device 12 as a separate device distinct from the computing device 12.

It is possible to generate plane wave reception signals at innumerable arbitrary plane wave angles or diverging wave reception signals for innumerable arbitrary virtual sound source locations, which can be obtained by the plane wave synthetic transmit focusing technique or the diverging wave synthetic transmit focusing technique by repeating the process of transmitting ultrasound with one ultrasound transducer element and receiving ultrasound with all ultrasound transducer elements to obtain and analyze a plurality of sets of received ultrasound data, thereby generating high-quality ultrasound images without reducing the frame rate.

The disclosed embodiments may be implemented in the form of a recording medium storing instructions executable by a computer. The instructions may be stored in the form of program codes, and when executed by a processor, may generate program modules to perform the operations of the disclosed embodiments. The recording medium may be implemented as a computer-readable recording medium.

Thus, the present disclosure has been described focusing on preferred embodiments thereof. Those skilled in the art will understand that the present disclosure may be implemented in modified forms without departing from the essential characteristics of the present invention. Therefore. the scope of the present invention is not limited to the embodiments described above, but should be construed to include various embodiments within the scope equivalent to the content described in the claims.

What is claimed is:

1. A method for generating an ultrasound image, the method comprising:
obtaining a plurality of sets of received ultrasound data by repeating a process of transmitting ultrasound to a target object with one ultrasound transducer element and receiving ultrasound reflected from the target object with all ultrasound transducer elements using an ultrasound probe including a plurality of ultrasound transducer elements; and
generating a plane wave synthetic focused image or a diverging wave synthetic focused image based on the plurality of sets of received ultrasound data.

2. The method for claim 1,
wherein the generating includes:
generating a plurality of plane wave reception signals that are obtained when plane waves are transmitted and received to and from the target object at various angles by analyzing the plurality of sets of received ultrasound data;
generating a plane wave synthetic signal by synthesizing the plurality of plane wave reception signals; and
generating a plane wave synthetic focused image based on the plane wave synthetic signal.

3. The method for claim 2,
wherein the generating of the plurality of plane wave reception signals includes:
calculating a transmission delay time and a reception delay time using Equations 1 and 2 below:
$${τ}_{tx} ( x , z , {x}_{n} , {θ}_{s} ) = \frac{( x - {x}_{n} ) sin {θ}_{s} + z cos {θ}_{s}}{c} \qquad [ Equation 1 ]$$
where, τₜₓ is a transmission delay time, x and z are the x-coordinate and z-coordinate of an image point, respectively, n is an index indicating an ultrasound transmission transducer element, xₙ is the x-coordinate of an ultrasound transmission transducer element n, s is an index indicating a plane wave angle, θₛ is a plane wave angle, and c is speed of sound; and
$${τ}_{rx} ( x , z , {x}_{m} ) = \frac{\sqrt{{( x - {x}_{m} )}^{2} + {z}^{2}}}{c} \qquad [ Equation 2 ]$$
where, τᵣₓ is the reception delay time, m is an index indicating an ultrasound reception transducer element, and xₘ is the x-coordinate of an ultrasound reception transducer element m; and
generating a plane wave reception signal having an arbitrary angle that are obtained when transmitting and receiving a plane wave to the target object at an arbitrary angle based on the transmission delay time and the reception delay time.

4. The method for claim 3,
wherein, in the generating of the plane wave reception signal having the arbitrary angle, the plane wave reception signal having the arbitrary angle is generated using Equations 3 and 4 below:
$$τ ( x , z , {x}_{n} , {x}_{m} , {θ}_{s} ) = {τ}_{tx} ( x , z , {x}_{n} , {θ}_{s} ) + {τ}_{rx} ( x , z , {x}_{m} ) \qquad [ Equation 3 ]$$
$${p}_{s} ( x , z ) = {\sum }_{n = 1}^{{N}_{iSF}} {\sum }_{m = 1}^{{N}_{t}} {r}_{n , m} ( τ ( x , z , {x}_{n} , {x}_{m} , {θ}_{s} ) ) \qquad [ Equation 4 ]$$
where, pₛ is a plane wave reception sound pressure signal, N_{iSF} is the number of ultrasound transmissions, Nₜ is the total number of ultrasound transducer elements used, and r_{n,m} is a received ultrasound signal obtained by the ultrasound reception transducer element m according to ultrasound transmission of the ultrasound transmission transducer element n.

5. The method for claim 4,
wherein, in the generating of the plane wave synthetic signal, the plane wave synthetic signal is generated using Equation 5 below:
$$p ( x , z ) = {\sum }_{s = 1}^{{N}_{θ}} {p}_{s} ( x , z ) = {\sum }_{s = 1}^{{N}_{θ}} {\sum }_{n = 1}^{{N}_{iSF}} {\sum }_{m = 1}^{{N}_{t}} {r}_{n , m} ( τ ( x , z , {x}_{n} , {x}_{m} , {θ}_{s} ) ) \qquad [ Equation 5 ]$$
where p is the plane wave synthetic signal, and N_{θ} is the total number of arbitrary plane wave angles to be synthesized.

6. The method for claim 1,
wherein the generating includes:
generating a plurality of diverging wave reception signals that are obtained when transmitting and receiving diverging waves generated from virtual sound sources at various locations to and from the target object by analyzing the plurality of sets of received ultrasound data;
generating a diverging wave synthetic signal by synthesizing the plurality of diverging wave reception signals; and
generating a diverging wave synthetic focused image based on the diverging wave synthetic signal.

7. The method for claim 6,
wherein the generating of the plurality of diverging wave reception signals includes:
calculating a transmission delay time and a reception delay time using Equations 6 and 7 below; and
generating a diverging wave reception signal at an arbitrary location that is obtained when transmitting and receiving a diverging wave generated from a virtual sound source at an arbitrary location to and from the target object based on the transmission delay time and the reception delay time:
$${τ}_{tx} ( x , z , {x}_{n} , {x}_{v , s} {z}_{v , s} ) = \frac{\sqrt{{( x - {x}_{v , s} )}^{2} + {( z - {z}_{v , s} )}^{2}} - \sqrt{{( {x}_{n} - {x}_{v , s} )}^{2} + {( {z}_{v , s} )}^{2}}}{c} \qquad [ Equation 6 ]$$
where, τₜₓ is a transmission delay time, x and z are the x-coordinate and z-coordinate of an image point, respectively, n is an index indicating an ultrasound transmission transducer element, xₙ is the x-coordinate of an ultrasound transmission transducer element n, s is an index indicating a virtual sound source location, x_{v,s} and z_{v,s} are the x-coordinate and the z-coordinate of the virtual sound source location s, respectively, and c is speed of sound; and
$${τ}_{rx} ( x , z , {x}_{m} ) = \frac{\sqrt{{( x - {x}_{m} )}^{2} + {z}^{2}}}{c} \qquad [ Equation 7 ]$$
wherein, τᵣₓ is a reception delay time, m is an index indicating an ultrasound reception transducer element, and xₘ is the x-coordinate of the ultrasound reception transducer element m.

8. The method for claim 7,
wherein, in the generating of the diverging wave reception signal at an arbitrary location, the diverging wave reception signal at the arbitrary location is generated using Equations 8 and 9 below:
$$τ ( x , z , {x}_{n} , {x}_{m} , {x}_{v , s} , {z}_{v , s} ) = {τ}_{tx} ( x , z , {x}_{n} , {x}_{v , s} , {z}_{v , s} ) + {τ}_{rx} ( x , z , {x}_{m} ) \qquad [ Equation 8 ]$$
$${p}_{s} ( x , z ) = {\sum }_{n = 1}^{{N}_{iSF}} {\sum }_{m = 1}^{{N}_{t}} {r}_{n , m} ( x , z , {x}_{n} , {x}_{m} , {x}_{v , s} , {z}_{v , s} ) \qquad [ Equation 9 ]$$
where, pₛ is the diverging wave reception signal, N_{iSF} is the number of ultrasound transmissions, Nₜ is the total number of ultrasound transducer elements used, and r_{n,m} is a received ultrasound signal obtained by the ultrasound reception transducer element m according to ultrasound transmission of the ultrasound transmission transducer element n.

9. The method for claim 8,
Wherein, in the generating of the diverging wave synthetic signal, the diverging wave synthetic signal is generated using Equation 10 below:
$$p ( x , z ) = {\sum }_{s = 1}^{{N}_{v}} {p}_{s} ( x , z ) = {\sum }_{s = 1}^{{N}_{v}} {\sum }_{n = 1}^{{N}_{iSF}} {\sum }_{m = 1}^{{N}_{t}} {r}_{n , m} ( x , z , {x}_{n} , {x}_{m} , {x}_{v , s} , {z}_{v , s} ) \qquad [ Equation 10 ]$$
where, p is the diverging wave synthesis signal, and N_{θ} is the total number of diverging waves to be synthesized.

10. An ultrasound imaging apparatus comprising:
an ultrasound probe including a plurality of ultrasound transducer elements, the ultrasound probe configured to obtain a plurality of sets of received ultrasound data by repeating a process of transmitting ultrasound to a target object with one ultrasound transducer element and receiving ultrasound reflected from the target object with all ultrasound transducer elements; and
an ultrasound image generator configured to generate a plane wave synthetic focused image or a diverging wave synthetic focused image based on the plurality of sets of received ultrasound data.

11. The ultrasound imaging apparatus of claim 10,
wherein the ultrasound image generator is configured to generate a plurality of plane wave reception signals that are obtained when plane waves are transmitted and received to and from the target object at various angles by analyzing the plurality of sets of received ultrasound data, generate a plane wave synthetic signal by synthesizing the plurality of plane wave reception signals, and generate a plane wave synthetic focused image based on the plane wave synthetic signal.

12. The ultrasound imaging apparatus of claim 11,
wherein the ultrasound image generator is configured to calculate a transmission delay time and a reception delay time using Equations 1 and 2 below and generate a plane wave reception signal having an arbitrary angle that is obtained when transmitting and receiving a plane wave to the target object at an arbitrary angle based on the transmission delay time and the reception delay time:
$${τ}_{tx} ( x , z , {x}_{n} , {θ}_{s} ) = \frac{( x - {x}_{n} ) sin {θ}_{s} + z cos {θ}_{s}}{c} \qquad [ Equation 1 ]$$
where, τₜₓ is a transmission delay time, x and z are the x-coordinate and z-coordinate of an image point, respectively, n is an index indicating an ultrasound transmission transducer element, xₙ is the x-coordinate of an ultrasound transmission transducer element n, s is an index indicating a plane wave angle, θₛ is a plane wave angle, and c is speed of sound; and
$${τ}_{rx} ( x , z , {x}_{m} ) = \frac{\sqrt{{( x - {x}_{m} )}^{2} + {z}^{2}}}{c} \qquad [ Equation 2 ]$$
where, τᵣₓ is the reception delay time, m is an index indicating an ultrasound reception transducer element, and xₘ is the x-coordinate of an ultrasound reception transducer element m.

13. The ultrasound imaging apparatus of claim 12,
wherein the ultrasound image generator is configured to generate the plane wave reception signal having the arbitrary angle using Equations 3 and 4 below:
$$τ ( x , z , {x}_{n} , {x}_{m} , {θ}_{s} ) = {τ}_{tx} ( x , z , {x}_{n} , {θ}_{s} ) + {τ}_{rx} ( x , z , {x}_{m} ) \qquad [ Equation 3 ]$$
$${p}_{s} ( x , z ) = {\sum }_{n = 1}^{{N}_{iSF}} {\sum }_{m = 1}^{{N}_{t}} {r}_{n , m} ( τ ( x , z , {x}_{n} , {x}_{m} , {θ}_{s} ) ) \qquad [ Equation 4 ]$$
where, pₛ is a plane wave reception sound pressure signal, N_{iSF} is the number of ultrasound transmissions, Nₜ is the total number of ultrasound transducer elements used, and r_{n,m} is a received ultrasound signal obtained by the ultrasound reception transducer element m according to ultrasound transmission of the ultrasound transmission transducer element n.

14. The ultrasound imaging apparatus of claim 13,
wherein the ultrasound image generator is configured to generate the plane wave synthetic signal using Equation 5 below:
$$p ( x , z ) = {\sum }_{s = 1}^{{N}_{θ}} {p}_{s} ( x , z ) = {\sum }_{s = 1}^{{N}_{θ}} {\sum }_{n = 1}^{{N}_{iSF}} {\sum }_{m = 1}^{{N}_{t}} {r}_{n , m} ( τ ( x , z , {x}_{n} , {x}_{m} , {θ}_{s} ) ) \qquad [ Equation 5 ]$$
where p is the plane wave synthetic signal, and N_{θ} is the total number of arbitrary plane wave angles to be synthesized.

15. The ultrasound imaging apparatus of claim 10,
wherein the ultrasound image generator is configured to generate a plurality of diverging wave reception signals that are obtained when transmitting and receiving diverging waves generated from virtual sound sources at various locations to and from the target object by analyzing the plurality of sets of received ultrasound data, generate a diverging wave synthetic signal by synthesizing the plurality of diverging wave reception signals, and generate a diverging wave synthetic focused image based on the diverging wave synthetic signal.

16. The ultrasound imaging apparatus of claim 15,
wherein the ultrasound image generator is configured to calculate a transmission delay time and a reception delay time using Equations 6 and 7 below and generate a diverging wave reception signal at an arbitrary location that is obtained when transmitting and receiving a diverging wave generated from a virtual sound source at an arbitrary location to and from the target object based on the transmission delay time and the reception delay time:
$${τ}_{tx} ( x , z , {x}_{n} , {x}_{v , s} {z}_{v , s} ) = \frac{\sqrt{{( x - {x}_{v , s} )}^{2} + {( z - {z}_{v , s} )}^{2}} - \sqrt{{( {x}_{n} - {x}_{v , s} )}^{2} + {( {z}_{v , s} )}^{2}}}{c} \qquad [ Equation 6 ]$$
where, τₜₓ is a transmission delay time, x and z are the x-coordinate and z-coordinate of an image point, respectively, n is an index indicating an ultrasound transmission transducer element, xₙ is the x-coordinate of an ultrasound transmission transducer element n, s is an index indicating a virtual sound source location, x_{v,s} and z_{v,s} are the x-coordinate and the z-coordinate of the virtual sound source location s, respectively, and c is speed of sound; and
$${τ}_{rx} ( x , z , {x}_{m} ) = \frac{\sqrt{{( x - {x}_{m} )}^{2} + {z}^{2}}}{c} \qquad [ Equation 7 ]$$
wherein, τᵣₓ is a reception delay time, m is an index indicating an ultrasound reception transducer element, and xₘ is the x-coordinate of the ultrasound reception transducer element m.

17. The ultrasound imaging apparatus of claim 16,
wherein the ultrasound image generator is configured to generate the diverging wave reception signal at the arbitrary location using Equations 8 and 9 below:
$$τ ( x , z , {x}_{n} , {x}_{m} , {x}_{v , s} , {z}_{v , s} ) = {τ}_{tx} ( x , z , {x}_{n} , {x}_{v , s} , {z}_{v , s} ) + {τ}_{rx} ( x , z , {x}_{m} ) \qquad [ Equation 8 ]$$
$${p}_{s} ( x , z ) = {\sum }_{n = 1}^{{N}_{iSF}} {\sum }_{m = 1}^{{N}_{t}} {r}_{n , m} ( x , z , {x}_{n} , {x}_{m} , {x}_{v , s} , {z}_{v , s} ) \qquad [ Equation 9 ]$$
where, pₛ is the diverging wave reception signal, N_{iSF} is the number of ultrasound transmissions, Nₜ is the total number of ultrasound transducer elements used, and r_{n,m} is a received ultrasound signal obtained by the ultrasound reception transducer element m according to ultrasound transmission of the ultrasound transmission transducer element n.

18. The ultrasound imaging apparatus of claim 17,
wherein the ultrasound image generator is configured to generate the diverging wave synthetic signal using Equation 10 below:
$$p ( x , z ) = {\sum }_{s = 1}^{{N}_{v}} {p}_{s} ( x , z ) = {\sum }_{s = 1}^{{N}_{v}} {\sum }_{n = 1}^{{N}_{iSF}} {\sum }_{m = 1}^{{N}_{t}} {r}_{n , m} ( x , z , {x}_{n} , {x}_{m} , {x}_{v , s} , {z}_{v , s} ) \qquad [ Equation 10 ]$$
where, p is the diverging wave synthesis signal, and N_{θ} is the total number of diverging waves to be synthesized.
